SEQUENCE LISTING (12 sequences)

SEQ ID NO: 1 - DNA, 23 bp, Artificial Sequence
  Forward outer primer for identifying beef
cttatattacgggtcttacactt

SEQ ID NO: 2 - DNA, 18 bp, Artificial Sequence
  Reverse outer primer for identifying beef
tgagaatccgcctcagat

SEQ ID NO: 3 - DNA, 50 bp, Artificial Sequence
  Forward inner primer for identifying beef
ggtaggacgtatcctatgaatgctgatgaaatattggagtaatccttctg

SEQ ID NO: 4 - DNA, 40 bp, Artificial Sequence
  Reverse inner primer for identifying beef
aggagcaacagtcatcaccagactaaatttgtgccgatgt

SEQ ID NO: 5 - DNA, 19 bp, Artificial Sequence
  Forward outer primer for identifying chicken
gccccatccaacatctctg

SEQ ID NO: 6 - DNA, 19 bp, Artificial Sequence
  Reverse outer primer for identifying chicken
cgtttgcgtggagattccg

SEQ ID NO: 7 - DNA, 42 bp, Artificial Sequence
  Forward inner primer for identifying chicken
atggctagtagtaggccggtgattcggctccctattagcagt

SEQ ID NO: 8 - DNA, 42 bp, Artificial Sequence
  Reverse inner primer for identifying chicken
cacagcagacacatccctagcctcagccgtattgtacgttcc

SEQ ID NO: 9 - DNA, 18 bp, Artificial Sequence
  Forward outer primer for identifying pork
attcattgacctcccagc

SEQ ID NO: 10 - DNA, 21 bp, Artificial Sequence
  Reverse outer primer for identifying pork
gtttgcatgtagatagcgaat

SEQ ID NO: 11 - DNA, 49 bp, Artificial Sequence
  Forward inner primer for identifying pork
gttaggatttgcaagattaggcagacaaacatctcatcatgatgaaact

SEQ ID NO: 12 - DNA, 45 bp, Artificial Sequence
  Reverse inner primer for identifying pork
agcaatacattacacatcagacacaatccgtaatttacgtctcga

(19) United States
(12) Patent Application Publication
(10) Pub. No.: US 2014/0193823 A1
(43) Pub. Date: Jul. 10, 2014

(54) PRIMER SET AND A METHOD FOR IDENTIFICATION OF MEAT SPECIES

(71) Applicant: NATIONAL PINGTUNG UNIVERSITY OF SCIENCE AND TECHNOLOGY, Pingtung County (TW)

(72) Inventors: Shyang-Chwen Sheu, Pingtung County (TW); Ke-Wei Chen, Pingtung County (TW)

(73) Assignee: NATIONAL PINGTUNG UNIVERSITY OF SCIENCE AND TECHNOLOGY, Pingtung County (TW)

(21) Appl. No.: 14/135,905

(22) Filed: Dec. 20, 2013

(30) Foreign Application Priority Data
Dec. 22, 2012 (TW) 101149394

(51) Int. Cl. C12Q 1/68

(52) U.S. Cl. CPC C12Q 1/6881
USPC 435/6.12

(57) ABSTRACT

The invention discloses a primer set for identification of meat species. The primer set comprises a pair of outer primers used to amplify a sense strand of DNA fragment of mitochondrial cytochrome b between positions 51 and 507. The pair of outer primers comprises a forward outer primer and a backward outer primer. The primer set further comprises a pair of inner primers comprising a forward inner primer and a backward inner primer. The forward inner primer comprises a first annealing portion and a first warped portion. The backward inner primer comprises a second annealing portion and a second warped portion. Accordingly, the primer set is used to identify species of meat especially with DNA fragmentation due to processing processes of processed food.

### BACKGROUND OF THE INVENTION

1. Field of the Invention

The present invention generally relates to a primer set for identifying meat species and a method using the primer set thereof and, more particularly, to a primer set amplifying a target fragment for identification of meat species and a method amplifying the target fragment with the primer set thereof

2. Description of the Related Art

For the safety issue, different cooking temperature is required for different meat species. For example, poultry should be cooked at a temperature higher than 75° C. to kill Campylobacter jejuni and bacteria belonging to Salmonella. Cooking at 56° C. for 5 minutes is required for beef to kill Taenia saginata. If any unexpected meat presents, the inadequate cooking temperature may result in unsufficient destroying of microorganisms or toxins. Therefore, the adulterated meat becomes a problem to food safety and sanitation. Moreover, some religion forbids eating of specific meat species. The adulterated meat may influence psychological status of the religion.

Some conventional methods for identification of meat species target specific proteins. These conventional methods comprise ELISA (enzyme-linked immunosorbent issay), SDS-PAGE (sodium dodecyl sulphate polyacrylamide gel electrophoresis) and Western blotting. However, the conventional methods targeting specific proteins are not suitable for identification of processed meat due to denaturation of proteins under processing process. Other conventional methods for identification of meat species including PCR (polymerase chain reaction), RFLP (restriction fragment length polymorphism), nested PCR (nested polymerase chain reaction) and real-time PCR (real-time polymerase chain reaction) amplify specific target fragments to identify meat species. The most broadly utilized conventional method is PCR. However, due to properties of polymerase used in PCR, PCR comprising several cycles at different temperature needs more operation time, thereby being not suitable for selection and detection of routine works. Moreover, Minhaz et al. (2010) provided another conventional method for identification of meat species, wherein a target fragment of mitochondrial 12S rRNA of a sample is amplified by Bst DNA polymerase at 63° C.

The conventional method reported by Minhaz et al. is suitable for identifying raw meat. However, the conventional method provided by Minhaz et al. uses a primer set targeting mitochondrial 12S rRNA, and the target fragment amplified by the Bst DNA polymerase is approximately 220 base pairs. Therefore, if the sample is a processed meat, DNA of the sample might be physically destroyed during processing process, thereby influencing precision of the conventional method provided by Minhaz et al. That is, the conventional method provided by Minhaz et al. has a narrow application.

In light of this, it is necessary to improve the conventional method for identification of meat species.

### SUMMARY OF THE INVENTION

It is therefore the objective of this invention to provide a primer set for identification of meat species, which is suitable for identifying any meat species with a great precision.

It is another objective of this invention to provide a method for identification of meat species with a great precision, thereby broadening application of the method for identification of meat species.

One embodiment of the invention discloses a primer set for identification of meat species comprises: a pair of outer primers used to amplify a sense strand of target fragment of mitochondrial cytochrome b between positions 51 and 507, comprising a forward outer primer and a backward outer primer, wherein the forward outer primer corresponds to a sense strand of DNA fragment of mitochondrial cytochrome b between positions 51 and 325, wherein the backward outer primer corresponds to an antisense strand of DNA fragment of mitochondrial cytochrome b between positions 235 and 507; and a pair of inner primers comprising a forward inner primer and a backward inner primer, wherein the forward inner primer comprises a first annealing portion and a first warped portion, wherein the first annealing portion corresponds to a sense DNA fragment of mitochondrial cytochrome b between positions 74 and 360, wherein the first warped portion corresponds to an antisense DNA fragment of mitochondrial cytochrome b between positions 116 and 401, the backward inner primer comprises a second annealing portion and a second warped portion, wherein the second annealing portion corresponds to a sense DNA fragment of mitochondrial cytochrome b between positions 153 and 442, wherein the second warped portion corresponds to an antisense DNA fragment of mitochondrial cytochrome b between positions 116 and 401.

In a preferred form shown, lengths of the forward outer primer and the backward outer primer are 18 to 23 bases.

In the preferred form shown, lengths of the first annealing portion, the first warped portion, the second annealing portion and the second warped portion are 20 to 25 bases.

In the preferred form shown, the forward outer primer corresponds to a sense strand of DNA fragment of mitochondrial cytochrome b of Bos indicus between positions 303 and 325, the backward outer primer corresponds to an antisense strand of DNA fragment of mitochondrial cytochrome b of Bos indicus between positions 490 and 507, the first annealing portion corresponds to a sense DNA fragment of mitochondrial cytochrome b of Bos indicus between positions 336 and 360, the first warped portion corresponds to an antisense DNA fragment of mitochondrial cytochrome b of Bos indicus between positions 377 and 401, the second annealing portion corresponds to a sense DNA fragment of mitochondrial cytochrome b of Bos indicus between positions 423 and 442, and the second warped portion corresponds to an antisense DNA fragment of mitochondrial cytochrome b of Bos indicus between positions 464 and 483.

In the preferred form shown, the forward outer primer has a sequence set forth in SEQ ID NO: 1, the backward outer primer has a sequence set forth in SEQ ID NO: 2, the forward inner primer has a sequence set forth in SEQ ID NO: 3, and the backward inner primer has a sequence set forth in SEQ ID NO: 4.

In the preferred form shown, the forward outer primer corresponds to a sense strand of DNA fragment of mitochondrial cytochrome b of Gallus gallus between positions 70 and 88, the backward outer primer corresponds to an antisense strand of DNA fragment of mitochondrial cytochrome b of Gallus gallus between positions 241 and 259, the first annealing portion corresponds to a sense DNA fragment of mitochondrial cytochrome b of Gallus gallus between positions 100 and 119, the first warped portion corresponds to an antisense DNA fragment of mitochondrial cytochrome b of Gallus gallus between positions 140 and 161, the second annealing portion corresponds to a sense DNA fragment of mitochondrial cytochrome b of Gallus gallus between positions 168 and 189, and the second warped portion corresponds to an antisense DNA fragment of mitochondrial cytochrome b of Gallus gallus between positions 215 and 234.

In the preferred form shown, the forward outer primer has a sequence set forth in SEQ ID NO: 5, the backward outer primer has a sequence set forth in SEQ ID NO: 6, the forward inner primer has a sequence set forth in SEQ ID NO: 7, and the backward inner primer has a sequence set forth in SEQ ID NO: 8.

In the preferred form shown, the forward outer primer corresponds to a sense strand of DNA fragment of mitochondrial cytochrome b of Sus scrofa domestica between positions 51 and 68, the backward outer primer corresponds to an antisense strand of DNA fragment of mitochondrial cytochrome b of Sus scrofa domestica between positions 235 and 255, the first annealing portion corresponds to a sense DNA fragment of mitochondrial cytochrome b of Sus scrofa domestica between positions 74 and 97, the first warped portion corresponds to an antisense DNA fragment of mitochondrial cytochrome b of Sus scrofa domestica between positions 116 and 140, the second annealing portion corresponds to a sense DNA fragment of mitochondrial cytochrome b of Sus scrofa domestica between positions 153 and 177, and the second warped portion corresponds to an antisense DNA fragment of mitochondrial cytochrome b of Sus scrofa domestica between positions 210 and 229.

In the preferred form shown, the forward outer primer has a sequence set forth in SEQ ID NO: 9, the backward outer primer has a sequence set forth in SEQ ID NO: 10, the forward inner primer has a sequence set forth in SEQ ID NO: 11, and the backward inner primer has a sequence set forth in SEQ ID NO: 12.

Another embodiment of the invention discloses a method for identification of meat species comprising: providing the primer set as mentioned above; mixing DNA template, a reaction buffer, dNTPs, the primer set and a Bst DNA polymerase to obtain a reaction mixture; amplifying a target fragment of mitochondrial cytochrome b of the DNA template of the reaction mixture at 65° C. to obtain an amplified target fragment; and analyzing the amplified target fragment by agarose electrophoresis.

In the preferred form shown, the forward outer primer has a sequence set forth in SEQ ID NO: 1, the backward outer primer has a sequence set forth in SEQ ID NO: 2, the forward inner primer has a sequence set forth in SEQ ID NO: 3, and the backward inner primer has a sequence set forth in SEQ ID NO: 4.

In the preferred form shown, the forward outer primer has a sequence set forth in SEQ ID NO: 5, the backward outer primer has a sequence set forth in SEQ ID NO: 6, the forward inner primer has a sequence set forth in SEQ ID NO: 7, and the backward inner primer has a sequence set forth in SEQ ID NO: 8.

In the preferred form shown, the forward outer primer has a sequence set forth in SEQ ID NO: 9, the backward outer primer has a sequence set forth in SEQ ID NO: 10, the forward inner primer has a sequence set forth in SEQ ID NO: 11, and the backward inner primer has a sequence set forth in SEQ ID NO: 12.

In the preferred form shown, the target fragment is amplified at 65° C. for 1 hour.

In the preferred form shown, the DNA template has an amount set forth in 1 to 100 ng.

In the preferred form shown, the primer set comprises the pair of outer primers being 0.5 μM and the pair of inner primers being 4 μM.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 is a schematic diagram demonstrating the primer set according to the invention annealing the DNA template comprising mitochondrial cytochrome b.

FIG. 2a shows integrity analyses of the DNA templates being extracted from raw beef, raw chicken and raw pork.

FIG. 2b shows integrity analyses of the DNA template being extracted from raw mutton, raw duck and raw goose.

FIG. 3a shows specificity analyses of the method for identification of meat species according to the first embodiment of the invention.

FIG. 3b shows specificity analyses of the method for identification of meat species according to the second embodiment of the invention.

FIG. 3c shows specificity analyses of the method for identification of meat species according to the third embodiment of the invention.

FIG. 4a shows sensitivity analyses of the method for identification of meat species according to the first embodiment of the invention.

FIG. 4b shows sensitivity analyses of the method for identification of meat species according to the second embodiment of the invention.

FIG. 4c shows sensitivity analyses of the method for identification of meat species according to the third embodiment of the invention.

FIG. 5a shows precision analyses of the method for identification of meat species according to the first embodiment of the invention used to identify raw beef mixture comprising raw mutton and raw pork.

FIG. 5b shows precision analyses of the method for identification of meat species according to the second embodiment of the invention used to identify raw chicken mixture comprising raw goose and raw duck.

FIG. 5c shows precision analyses of the method for identification of meat species according to the third embodiment of the invention used to identify raw pork mixture comprising raw beef and raw mutton.

FIG. 6a shows results of the method for identification of meat species according to the first embodiment of the invention used to identify boiled beef.

FIG. 6b shows results of the method for identification of meat species according to the second embodiment of the invention used to identify boiled chicken.

FIG. 6c shows results of the method for identification of meat species according to the third embodiment of the invention used to identify boiled pork.

FIG. 7a shows results of the method for identification of meat species according to the first embodiment of the invention used to identify pressure-cooked beef

FIG. 7b shows results of the method for identification of meat species according to the second embodiment of the invention used to identify pressure-cooked chicken.

FIG. 7c shows results of the method for identification of meat species according to the third embodiment of the invention used to identify pressure-cooked pork.

FIG. 8a shows results of the method for identification of meat species according to the first embodiment of the invention used to identify commercially available processed food comprising beef.

FIG. 8b shows results of the method for identification of meat species according to the second embodiment of the invention used to identify commercially available processed food comprising chicken.

FIG. 8c shows results of the method for identification of meat species according to the third embodiment of the invention used to identify commercially available processed food comprising pork.

In the various figures of the drawings, the same numerals designate the same or similar parts. Furthermore, when the term “first”, “second”, “third”, “fourth”, “inner”, “outer” “top”, “bottom” and similar terms are used hereinafter, it should be understood that these terms refer only to the structure shown in the drawings as it would appear to a person viewing the drawings, and are utilized only to facilitate describing the invention.

### DETAILED DESCRIPTION OF THE INVENTION

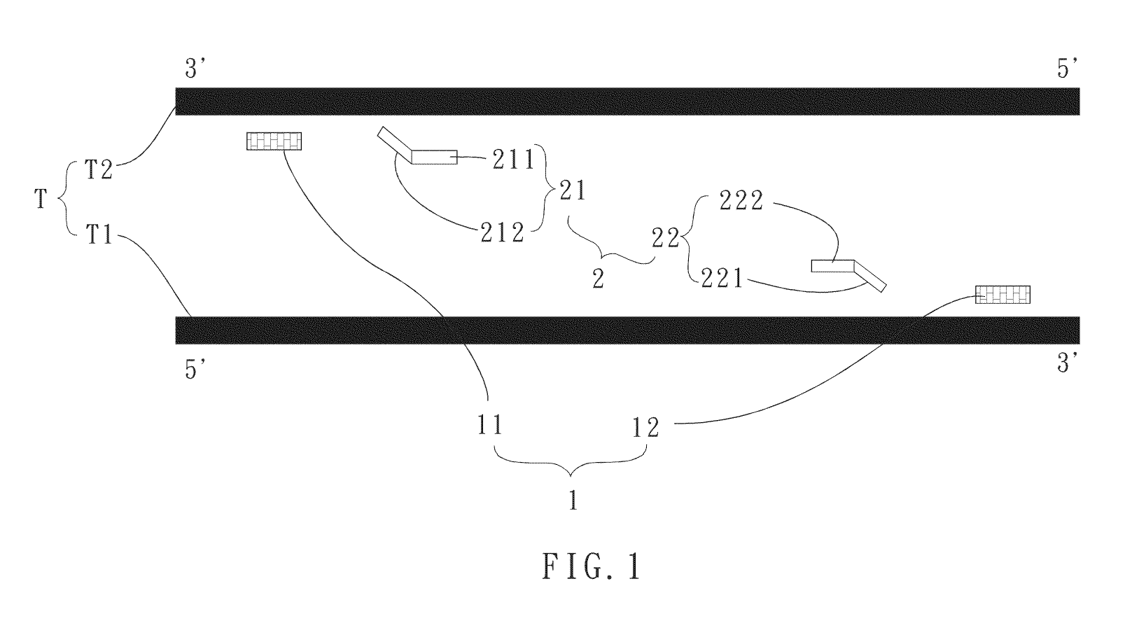

Referring to FIG. 1 being a schematic diagram, which demonstrates a primer set for identification of meat species according to the invention annealing DNA template “T” comprising mitochondrial cytochrome b gene. The DNA template “T”, with mitochondrial cytochrome b, comprises a sense strand T1 and an antisense strand T2. The primer set for identifying meat species comprises a pair of outer primers 1 and a pair of inner primers 2. The pair of outer primers 1, comprising a forward outer primer 11 and a backward outer primer 12, are used to amplify a sense strand of DNA fragment of mitochondrial cytochrome b between positions 51 and 507. The pair of inner primers 2 comprises a forward inner primer 21 and a backward inner primer 22. According to sequences of the forward inner primer 21 and the backward inner primer 22, an amplified target fragment may form various sized structures consisting of alternatively inverted repeats.

In detail, the forward outer primer 11 corresponds to a sense strand of DNA fragment of mitochondrial cytochrome b between positions 51 and 235, while the backward outer primer 12 corresponds to an antisense strand of DNA fragment of mitochondrial cytochrome b between positions 235 and 507. In the preferred embodiment, lengths of the forward outer primer and the backward outer primer are 18 to 23 bases.

The forward inner primer 21 has a first annealing portion 211 and a first warped portion 212, wherein the first annealing portion 211 corresponds to a sense strand of DNA fragment of mitochondrial cytochrome b between positions 75 and 360, wherein the first warped portion 213 corresponds to a antisense strand of DNA fragment of mitochondrial cytochrome b between positions 116 and 401. In addition, the backward inner primer 22 has a second annealing portion 221 and a second warped portion 222, wherein the second annealing portion corresponds to a sense strand of DNA fragment of mitochondrial cytochrome b between positions 155 and 442, wherein the second warped portion 222 corresponds to an antisense strand of DNA fragment of mitochondrial cytochrome b between positions 116 and 401. In the preferred embodiment, lengths of the forward inner primer and the backward inner primer are 40 to 50 bases, with lengths of the first annealing portion and the second annealing portion being 20 to 25 bases, with lengths of the first warped portion and the second warped portion being 20 to 25 bases.

The primer set according to the invention is able to specifically amplify the target fragment of mitochondrial cytochrome b of various meat species, such as beef, chicken and pork. In detail, mitochondrial cytochrome b is a locus of mitochondrial DNA. Mitochondrial DNA has a relatively greater copy numbers compared with nuclear DNA, thereby showing higher success rate especially with regard to DNA templates containing high fragmentation, for example, the DNA templates extracted form processed meat. DNA sequences of mitochondrial cytochrome b are highly conserved among a same species while show apparent difference between different species, thereby being suitable for identifying what species the DNA template belongs to.

The primer set according to the first embodiment of the invention is designed according to a DNA sequence of GenBank database being mitochondrial cytochrome b of Bos indicus with a sequence number being GU256940. That is, the primer set is capable of amplifying a target fragment of mitochondrial cytochrome b of Bos indicus. The forward outer primer 11, with length being 23 bases, corresponds to a sense strand of DNA fragment of mitochondrial cytochrome b of Bos indicus between positions 303 and 325. The backward outer primer 12, with length being 18 bases, corresponds to an antisense strand of DNA fragment of mitochondrial cytochrome b of Bos indicus between positions 490 and 507. The first annealing portion 211, with length being 25 bases, corresponds to a sense strand of DNA fragment of mitochondrial cytochrome b of Bos indicus between positions 336 and 360, while the first warped portion 212, with length being 25 bases, corresponds to an antisense strand of DNA fragment of mitochondrial cytochrome b of Bos indicus between positions 337 and 401. The second annealing portion 221, with length being 20 bases, corresponds to a sense strand of DNA fragment of mitochondrial cytochrome b of Bos indicus between positions 423 and 442, while the second warped portion 222, with length being 20 bases, corresponds to an antisense strand of DNA fragment of mitochondrial cytochrome b of Bos indicus between positions 464 and 483. Preferably, the forward outer primer 11 comprises a sequence set forth in SEQ ID NO: 1, the backward outer primer 12 comprises a sequence set forth in SEQ ID NO: 2, the forward inner primer 21 comprises a sequence set forth in SEQ ID NO: 3, and the backward inner primer 22 comprises a sequence set forth in SEQ ID NO: 4.

The primer set according to the second embodiment of the invention is designed according to a DNA sequence of GenBank database being mitochondrial cytochrome b of Gallus gallus with a sequence number being AF119093. That is, the primer set is capable of amplifying a target fragment of mitochondrial cytochrome b of Gallus gallus. The forward outer primer 11, with length being 19 bases, corresponds to a sense strand of DNA fragment of mitochondrial cytochrome b of Gallus gallus between positions 70 and 88. The backward outer primer 12, with length being 19 bases, corresponds to an antisense strand of DNA fragment of mitochondrial cytochrome b of Gallus gallus between positions 241 and 259. The first annealing portion 211, with length being 20 bases, corresponds to a sense strand of DNA fragment of mitochondrial cytochrome b of Gallus gallus between positions 100 and 119, while the first warped portion 212, with length being 21 bases, corresponds to an antisense strand of DNA fragment of mitochondrial cytochrome b of Gallus gallus between positions 140 and 161. The second annealing portion 221, with length being 22 bases, corresponds to a sense strand of DNA fragment of mitochondrial cytochrome b of Gallus gallus between positions 168 and 189, while the second warped portion 222, with length being 20 bases, corresponds to an antisense fragment of mitochondrial cytochrome b of Gallus gallus between positions 215 and 234. Preferably, the forward outer primer 11 comprises a sequence set forth in SEQ ID NO: 5, the backward outer primer 12 comprises a sequence set forth in SEQ ID NO: 6, the forward inner primer 21 comprises a sequence set forth in SEQ ID NO: 7, and the backward inner primer 22 comprises a sequence set forth in SEQ ID NO: 8.

The primer set according to the third embodiment of the invention is designed according to a DNA sequence of GenBank database being mitochondrial cytochrome b of Sus scrofa with sequence number being GU211931. That is, the primer set is capable of amplifying a target fragment of mitochondrial cytochrome b of Sus scrofa. The forward outer primer 11, with length being 18 bases, corresponds to a sense strand of DNA fragment of mitochondrial cytochrome b of Sus scrofa between positions 51 and 68. The backward outer primer 12, with length being 21 bases, corresponds to an antisense strand of DNA fragment of mitochondrial cytochrome b of Sus scrofa between positions 235 and 255. The first annealing portion 211, with length being 24 bases, corresponds to a sense strand of DNA fragment of mitochondrial cytochrome b of Sus scrofa between positions 74 and 97, while the first warped portion 212, with length being 25 bases, corresponds to an antisense strand of DNA fragment of mitochondrial cytochrome b of Sus scrofa between positions 116 and 140. The second annealing portion 221, with length being 25 bases, corresponds to a sense strand of DNA fragment of mitochondrial cytochrome b of Sus scrofa between positions 153 and 177, while the second warped portion 222, with length being 20 bases, corresponds to an antisense strand of DNA fragment of mitochondrial cytochrome b of Sus scrofa between positions 210 and 229. Preferably, the forward outer primer 11 comprises a sequence set forth in SEQ ID NO: 9, the backward outer primer 12 comprises a sequence set forth in SEQ ID NO: 10, the forward inner primer 21 comprises a sequence set forth in SEQ ID NO: 11, and the backward inner primer 22 comprises a sequence set forth in SEQ ID NO: 12.

Besides, a method for identification of meat species according to the invention comprises: providing the primer set as mentioned above with the pair of outer primers 1 and the pair of inner primers 2, mixing with DNA template, a reaction buffer, dNTPs and a Bst DNA polymerase to obtain a reaction mixture, amplifying a target fragment of mitochondrial cytochrome b of the DNA template at 65° C. to obtain an amplified target fragment, and analyzing the amplified target fragment of mitochondrial cytochrome b by agarose electrophoresis.

Further, according to target meat species, the primer set can choose to be the primer set according to the first, second or third embodiment of the invention. For example, when the target meat species is beef (Bos indicus), the primer set is set forth being the primer set according to the first embodiment of the invention, wherein the forward outer primer 11 comprises the sequence set forth in SEQ ID NO: 1, wherein the backward outer primer 12 comprises the sequence set forth in SEQ ID NO: 2, wherein the forward inner primer 21 comprises the sequence set forth in SEQ ID NO: 3, wherein the backward inner primer 22 comprises the sequence set forth in SEQ ID NO: 4. Moreover, when the target meat species is chicken (Gallus gallus), the primer set is set forth being the primer set according to the second embodiment of the invention, wherein the forward outer primer 11 comprises the sequence set forth in SEQ ID NO: 5, wherein the backward outer primer 12 comprises the sequence set forth in SEQ ID NO: 6, wherein the forward inner primer 21 comprises the sequence set forth in SEQ ID NO: 7, wherein the backward inner primer 22 comprises the sequence set forth in SEQ ID NO: 8. Furthermore, when the target meat species is pork (Sus scrofa), the primer set is set forth being the primer set according to the third embodiment of the invention, wherein the forward outer primer 11 comprises the sequence set forth in SEQ ID NO: 9, wherein the backward outer primer 12 comprises the sequence set forth in SEQ ID NO: 10, wherein the forward inner primer 21 comprises the sequence set forth in SEQ ID NO: 11, wherein the backward inner primer 22 comprises the sequence set forth in SEQ ID NO: 12.

For instance, the method for identification of meat species according to the first, second and third embodiments of the invention comprise providing the DNA template. The DNA template may be extracted from raw meat, cooked meat or processed meat. Alternatively, the DNA template may be extracted from animal tissues such as muscles, organs or bloods. Preferably, the DNA template is mitochondrial DNA.

In detail, the DNA template is extracted but not limited as followed: providing an adequate amount of a sample, such as 1 gram of a raw meat, mixing the sample with 4 mL of TNES (20 mM Tris, 150 mM NaCl, 10 mM ETDA, pH 8.0), 50 μL of protease K (20 mg/mL) and 250 μL of 10% SDS in a 15 mL tube and digesting the sample at 56° C. for 8 to 12 hours to obtain a first supernatant. 750 μL of the first supernatant mixing with 30 μL of Rnase (10 mg/mL) is digested at 37° C. for 1 hour, followed by mixing with 250 μL of saturated NaCl solution and centrifugation for 10 minutes (5,400×g, 4° C.) to obtain a second supernatant. The second supernatant mixes with PCI (phenol:chloroform:isoamyl alcohol=25:24:1) with a volumetric ratio of the second supernatant and the PCI being 1:1, followed by centrifugation at 6,400×g for 10 minutes at 4° C. to obtain a third supernatant. The third supernatant mixes with chloroform with a volumetric ratio of the third supernatant and chloroform being 1:1, followed by centrifugation 6,400×g for 10 minutes at 4° C. to obtain a fourth supernatant. The fourth supernatant mixes with 100% ethanol (−20° C.) and 30 μL of sodium acetate (0.1 M, pH 5.0) with a volumetric ratio of the fourth supernatant and ethanol being 1:2, followed by standing at −80° C. for 2 hours, centrifugation 7,600×g for 10 minutes at 4° C. to obtain a pellet. The pellet is washed by 300 μL of 70% ethanol to remove salts on the pellet, followed by centrifugation for 5 minutes at room temperature, discarding 70% ethanol, and volatilizing the 70% ethanol for 15 minutes at room temperature to obtain the DNA sample. The DNA sample can be further dissolved in 50 μL sterile water to obtain a DNA sample solution. The DNA sample solution can be stored at −20° C. for further use as the DNA template.

An adequate amount of the DNA template is formulated the reaction mixture as shown in Table 1 in a 0.2 mL PCR reaction tube, wherein preferably the DNA template of the reaction mixture is between 1 and 100 ng.

TABLE 1
Formulation of the reaction mixture

| Formulation | Amounts | |
|---|---|---|
| DNA template | adequate | |
| Tris-HCl (pH 9.0) | 200 | mM |
| MgSO₄ | 20 | mM |
| KCl | 100 | mM |
| (NH₄)₂SO₄ | 100 | mM |
| Triton X-100 | 0.1% | |
| DNTPs | 200 | μM |
| the pair of outer primers 1 | 0.5 | μM |
| the pair of inner primers 2 | 4 | μM |
| Bst polymerase | 1 | U |
| sterile water | to 25 | μL |

The reaction mixture subsequently stands at 65° C. for 1 hour to perform an amplification reaction, wherein the target fragment of mitochondrial cytochrome b of the DNA template is specifically amplified by the primer set. Preferably, the 0.2 mL PCR reaction tube containing the reaction mixture can be placed in a PCR reactor. Furthermore, after the amplification reaction, the Bst polymerase is inactivated at 80° C. for 5 minutes. Finally, the reaction mixture finishing the amplification reaction can be stored at 4° C.

The amplified target fragment of mitochondrial cytochrome b is analyzed by agarose electrophoresis. That is, the reaction mixture finishing the amplification reaction may mix with a 6× loading dye (with 0.25% bromophenol blue, 0.25% xylene cyanol FF and 30% glycerol) and further be analyzed by a 1% agarose gel soaked in 0.5×TBE buffer under 100 volts, followed by staining with EtBr. The stained agarose gel is destained by running water for 10 to 12 minutes, and a result of the destained agarose gel is analyzed under UV light of 314 nm.

If the target fragment of mitochondrial cytochrome b of the DNA template can be specifically amplified by the primer set, a characteristic ladder pattern is observed on the destained agarose gel. In contrary, if the target fragment of mitochondrial cytochrome b of the DNA template cannot be amplified by the primer set, the characteristic ladder pattern does not appear on the destained agarose gel. Accordingly, one of ordinary skill in the art would assuredly understand whether the sample contains the meat species desired to identify.

In order to prove the method for identifying meat species according to the embodiment of the invention can be used to identify the samples being beef, chicken or pork, and to prove the method for identifying meat species according to the embodiment of the invention can be used to identify the samples being mixtures or processed meat, several trials have been performed as follows:

Trial (a). Extraction of the DNA Template and Following Quantitative Analysis

Sources of the DNA templates used in the trail (A) are shown in Table 2. 1 gram of the samples is extracted using the method mentioned above to obtain the DNA sample solution, respectively. Further, 5 μL of the DNA sample solution mixes with 495 μL of sterile DEPC water, followed by measuring OD₂₆₀ and OD₂₈₀ using a photocytometer. Purifies of the DNA templates are measured according to a ratio of OD₂₆₀/OD₂₈₀.

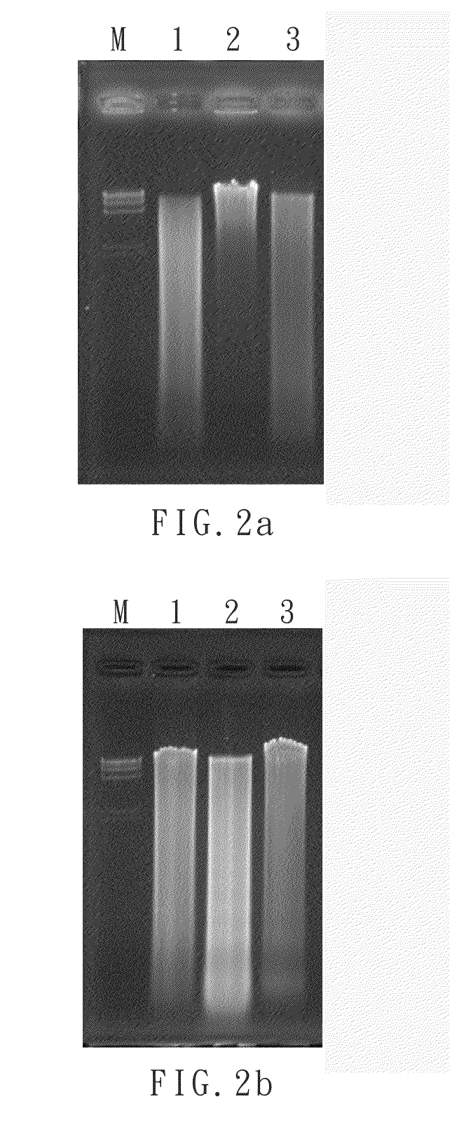

TABLE 2
The samples used in the trail (A)

| | | Sources of the | |
|---|---|---|---|
| Groups | FIGS. | DNA templates | OD₂₆₀/OD₂₈₀ |
| A1-1 | FIG. 2a lane 1 | Raw beef | 1.88 |
| A1-2 | FIG. 2a lane 2 | Raw chicken | 1.79 |
| A1-3 | FIG. 2a lane 3 | Raw pork | 1.80 |
| A2-1 | FIG. 2b lane 1 | Raw mutton | 1.75 |
| A2-2 | FIG. 2b lane 2 | Raw duck | 1.82 |
| A2-3 | FIG. 2b lane 3 | Raw goose | 1.74 |

5 μL of the DNA sample solutions mix with 1 μL of 6× loading dye, followed by analyzing using the 1% agarose gel soaked in 0.5×TBE buffer under 100 volts. After staining with EtBr and destained with running water for 10 to 12 minutes, the destained agarose gel is analyzed under UV light of 314 nm

FIGS. 2a and 2b show results of DNA templates being extracted from raw beef, raw chicken, raw pork, raw mutton, raw duck and raw goose. All of the DNA templates have great integrity with no fragmentation.

Trial (B). Specificity Test of the Primer Sets

Sources of the DNA templates and the primer sets used in the trail (B) are shown in Table 3. The reaction mixtures containing 100 ng of the DNA templates, respectively are heated at 65° C. for 1 hour, followed by inactivating the Bst polymerase at 80° C. for 5 minutes. The reaction mixtures store at 4° C. for further use.

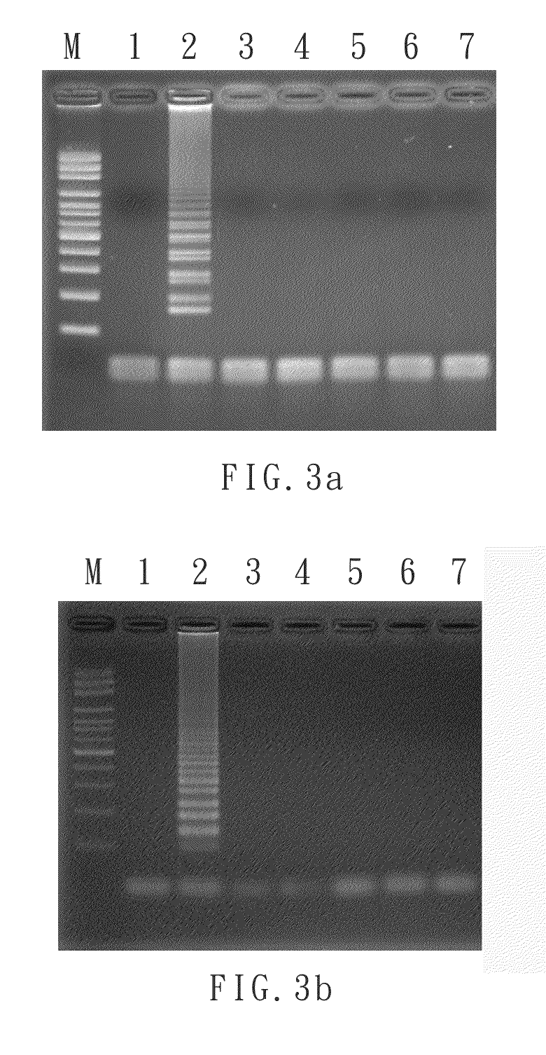

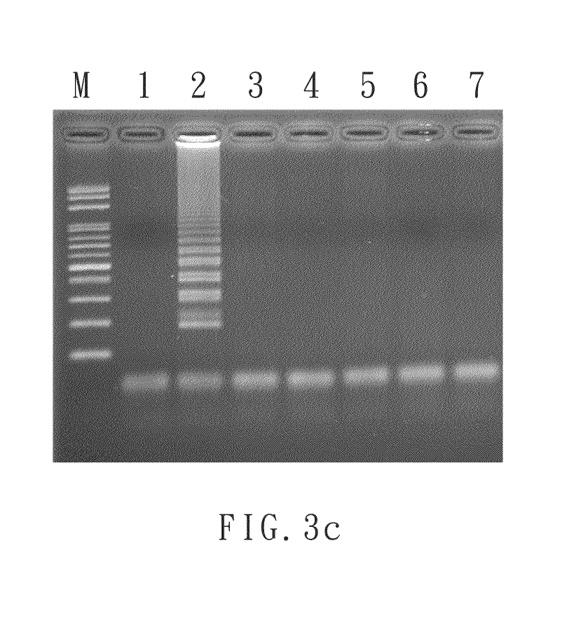

TABLE 3
The DNA templates and the primer pair used in the trial (B)

| | | Sources of the | |
|---|---|---|---|
| Groups | FIGS. | DNA templates | Primer sets |
| B1-1 | FIG. 3a lane 1 | — | Forward outer |
| B1-2 | FIG. 3a lane 2 | Raw beef | primer: SEQ ID |
| B1-3 | FIG. 3a lane 3 | Raw pork | NO: 1 |
| B1-4 | FIG. 3a lane 4 | Raw chicken | Backward outer |
| B1-5 | FIG. 3a lane 5 | Raw mutton | primer: SEQ ID |
| B1-6 | FIG. 3a lane 6 | Raw duck | NO: 2 |
| B1-7 | FIG. 3a lane 7 | Raw goose | Forward inner |
| | | | primer: SEQ ID |
| | | | NO: 3 |
| | | | Backward inner |
| | | | primer: SEQ ID |
| | | | NO: 4 |
| B2-1 | FIG. 3b lane 1 | — | Forward outer |
| B2-2 | FIG. 3b lane 2 | Raw chicken | primer: SEQ ID |
| B2-3 | FIG. 3b lane 3 | Raw beef | NO: 5 |
| B2-4 | FIG. 3b lane 4 | Raw pork | Backward outer |
| B2-5 | FIG. 3b lane 5 | Raw mutton | primer: SEQ ID |
| B2-6 | FIG. 3b lane 6 | Raw duck | NO: 6 |
| B2-7 | FIG. 3b lane 7 | Raw goose | Forward inner |
| | | | primer: SEQ ID |
| | | | NO: 7 |
| | | | Backward inner |
| | | | primer: SEQ ID |
| | | | NO: 8 |
| B3-1 | FIG. 3c lane 1 | — | Forward outer |
| B3-2 | FIG. 3c lane 2 | Raw pork | primer: SEQ ID |
| B3-3 | FIG. 3c lane 3 | Raw beef | NO: 9 |
| B3-4 | FIG. 3c lane 4 | Raw chicken | Backward outer |
| B3-5 | FIG. 3c lane 5 | Raw duck | primer: SEQ ID |
| B3-6 | FIG. 3c lane 6 | Raw mutton | NO: 10 |
| B3-7 | FIG. 3c lane 7 | Raw goose | Forward inner |
| | | | primer: SEQ ID |
| | | | NO: 11 |
| | | | Backward inner |
| | | | primer: SEQ ID |
| | | | NO: 12 |

Referring to FIG. 3a lane 2, the primer set according to the first embodiment of the invention shows the characteristic ladder pattern on the 1% agarose gel. However, none target species show no the characteristic ladder pattern on the 1% agarose gel as shown in FIG. 3a lanes 3 to 7. Similar results are shown in FIGS. 3b and 3c as the primer sets being in accordance with the second and third embodiment of the invention, respectively. As a result, the primer sets according to the first, second and third embodiments show high specificity for target meat species.

Trial (C). Limit Test of the Primer Sets

Sources of the DNA templates with different amounts and the primer sets used in the trail (C) are shown in Table 4. The method for identification of meat species is the same as above.

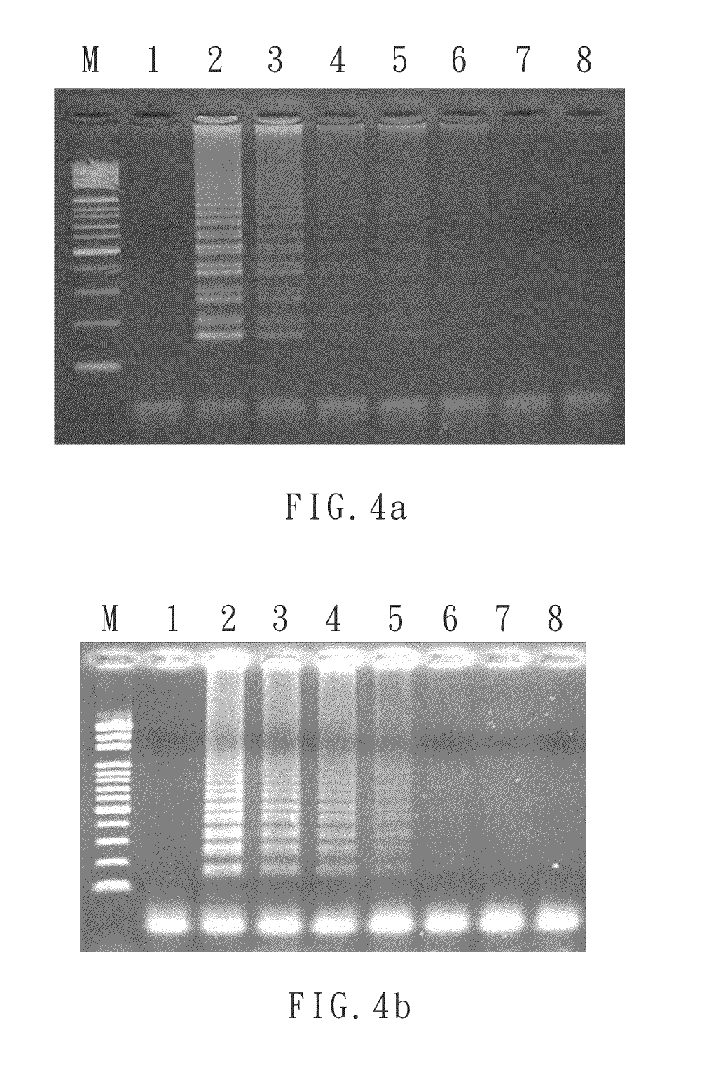

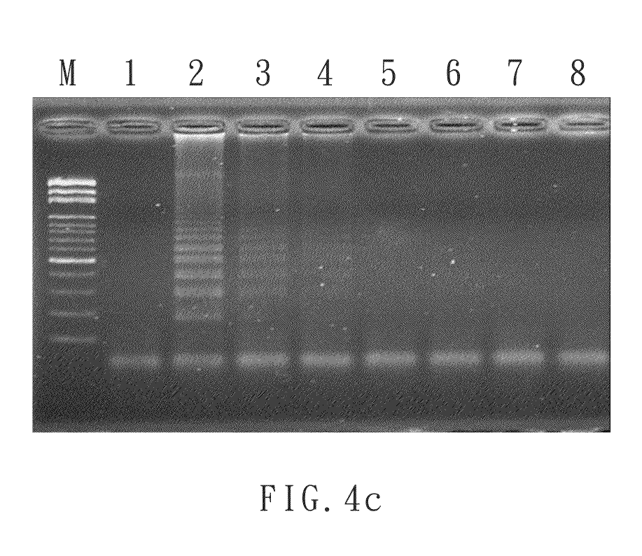

TABLE 4
The DNA templates and the primer pair used in the trial (C)

| | | DNA templates | | |
|---|---|---|---|---|
| Groups | FIGS. | Sources | Amounts (ng) | Primer sets |
| C1-1 | FIG. 4a lane 1 | — | — | Forward outer |
| C1-2 | FIG. 4a lane 2 | Raw beef | 10² | primer: SEQ ID |
| C1-3 | FIG. 4a lane 3 | Raw beef | 10¹ | NO: 1 |
| C1-4 | FIG. 4a lane 4 | Raw beef | 10⁰ | Backward outer |
| C1-5 | FIG. 4a lane 5 | Raw beef | 10⁻¹ | primer: SEQ ID |
| C1-6 | FIG. 4a lane 6 | Raw beef | 10⁻² | NO: 2 |
| C1-7 | FIG. 4a lane 7 | Raw beef | 10⁻³ | Forward inner |
| C1-8 | FIG. 4a lane 8 | Raw beef | 10⁻⁴ | primer: SEQ ID |
| | | | | NO: 3 |
| | | | | Backward inner |
| | | | | primer: SEQ ID |
| | | | | NO: 4 |
| C2-1 | FIG. 4b lane 1 | — | — | Forward outer |
| C2-2 | FIG. 4b lane 2 | Raw | 10² | primer: SEQ ID |
| | | chicken | | NO: 5 |
| C2-3 | FIG. 4b lane 3 | Raw | 10¹ | Backward outer |
| | | chicken | | primer: SEQ ID |
| C2-4 | FIG. 4b lane 4 | Raw | 10⁰ | NO: 6 |
| | | chicken | | Forward inner |
| C2-5 | FIG. 4b lane 5 | Raw | 10⁻¹ | primer: SEQ ID |
| | | chicken | | NO: 7 |
| C2-6 | FIG. 4b lane 6 | Raw | 10⁻² | Backward inner |
| | | chicken | | primer: SEQ ID |
| C2-7 | FIG. 4b lane 7 | Raw | 10⁻³ | NO: 8 |
| | | chicken | | |
| C2-8 | FIG. 4b lane 8 | Raw | 10⁻⁴ | |
| | | chicken | | |
| C3-1 | FIG. 4c lane 1 | — | — | Forward outer |
| C3-2 | FIG. 4c lane 2 | Raw pork | 10² | primer: SEQ ID |
| C3-3 | FIG. 4c lane 3 | Raw pork | 10¹ | NO: 9 |
| C3-4 | FIG. 4c lane 4 | Raw pork | 10⁰ | Backward outer |
| C3-5 | FIG. 4c lane 5 | Raw pork | 10⁻¹ | primer: SEQ ID |
| C3-6 | FIG. 4c lane 6 | Raw pork | 10⁻² | NO: 10 |
| C3-7 | FIG. 4c lane 7 | Raw pork | 10⁻³ | Forward inner |
| C3-8 | FIG. 4c lane 8 | Raw pork | 10⁻⁴ | primer: SEQ ID |
| | | | | NO: 11 |
| | | | | Backward inner |
| | | | | primer: SEQ ID |
| | | | | NO: 12 |

Referring to FIGS. 4a, 4b and 4c, the lower concentration is, the weaker the characteristic ladder pattern is. In FIG. 4a, lanes 2 to 6 with sample DNA amounts being 100 to 0.01 ng show the characteristic ladder pattern. In FIG. 4b, lanes 2 to 6 with sample DNA amounts being 100 to 0.01 ng also show the characteristic ladder pattern. In FIG. 4c, lanes 2 to 4 with sample DNA amounts being 100 to 1 ng show the characteristic ladder pattern. That is, the primer sets according to the first, second and third embodiments of the invention have limits of amounts of the DNA templates being 0.01, 0.01 and 1 ng, respectively.

Trial (D). Identification Test of the Sample being Mixtures

The DNA templates are extracted from raw meat being mixtures selected from a group consisting of beef, chicken, pork, mutton, duck and goose with a ratio shown in Table 5. Further, the primer sets used in the trail (D) are also shown in Table 5. The method for identification of meat species is the same as above.

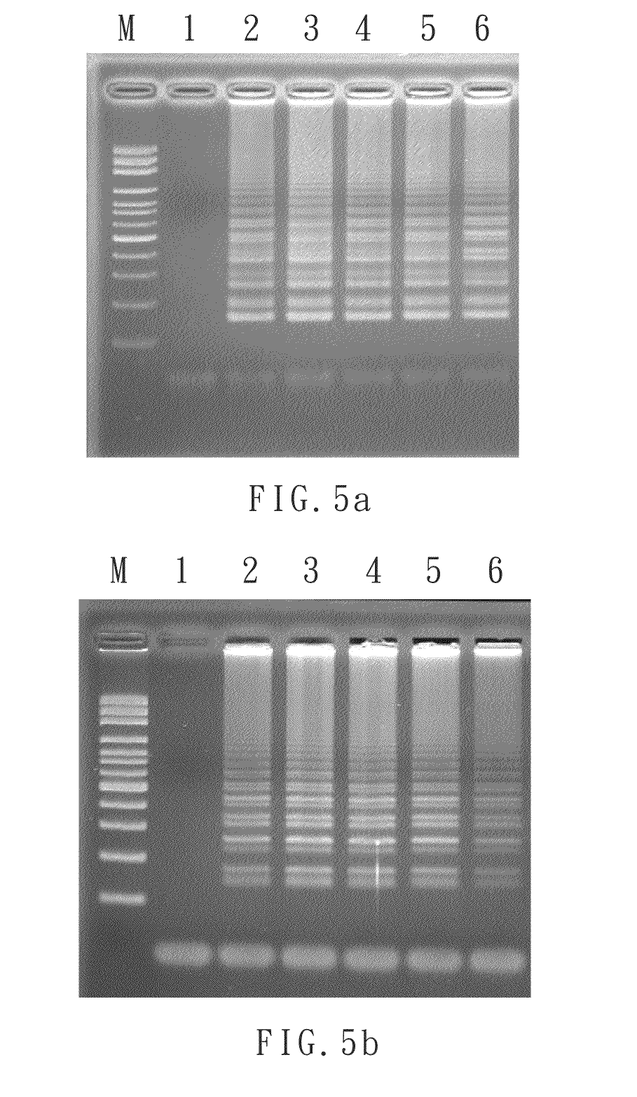

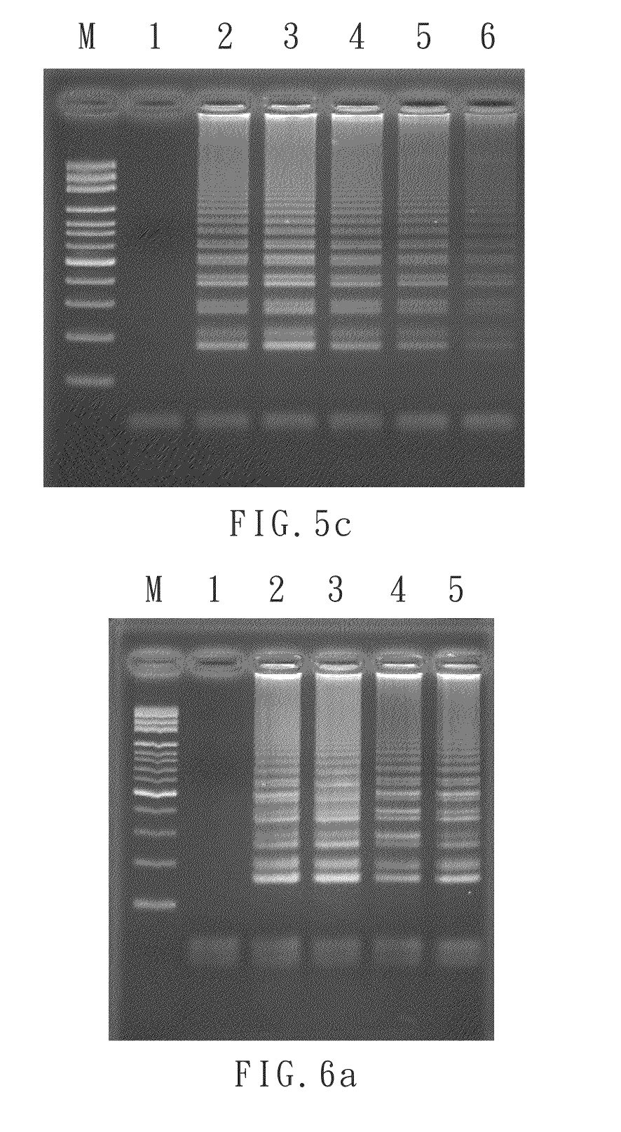

TABLE 5
The DNA templates with different ratio of raw meats and the
primer pair used in the trial (D)

| | | Ratio (wt %) | | | | | | |
|---|---|---|---|---|---|---|---|---|
| Groups | Figures | Beef | Mutton | Pork | Chicken | Duck | Goose | Primer sets |
| D1-1 | FIG. 5a | 0 | 0 | 0 | 0 | 0 | 0 | Forward |
| | lane 1 | | | | | | | outer |
| D1-2 | FIG. 5a | 100 | 0 | 0 | 0 | 0 | 0 | primer: |
| | lane 2 | | | | | | | SEQ ID |
| D1-3 | FIG. 5a | 50 | 25 | 25 | 0 | 0 | 0 | NO: 1 |
| | lane 3 | | | | | | | Backward |
| D1-4 | FIG. 5a | 10 | 45 | 45 | 0 | 0 | 0 | outer |
| | lane 4 | | | | | | | primer: |
| D1-5 | FIG. 5a | 5 | 47.5 | 47.5 | 0 | 0 | 0 | SEQ ID |
| | lane 5 | | | | | | | NO: 2 |
| D1-6 | FIG. 5a | 1 | 49.5 | 49.5 | 0 | 0 | 0 | Forward |
| | lane 6 | | | | | | | inner |
| | | | | | | | | primer: |
| | | | | | | | | SEQ ID |
| | | | | | | | | NO: 3 |
| | | | | | | | | Backward |
| | | | | | | | | inner |
| | | | | | | | | primer: |
| | | | | | | | | SEQ ID |
| | | | | | | | | NO: 4 |
| D2-1 | FIG. 5b | 0 | 0 | 0 | 0 | 0 | 0 | Forward |
| | lane 1 | | | | | | | outer |
| D2-2 | FIG. 5b | 0 | 0 | 0 | 100 | 0 | 0 | primer: |
| | lane 2 | | | | | | | SEQ ID |
| D2-3 | FIG. 5b | 0 | 0 | 0 | 50 | 25 | 25 | NO: 5 |
| | lane 3 | | | | | | | Backward |
| D2-4 | FIG. 5b | 0 | 0 | 0 | 10 | 45 | 45 | outer |
| | lane 4 | | | | | | | primer: |
| D2-5 | FIG. 5b | 0 | 0 | 0 | 5 | 47.5 | 47.5 | SEQ ID |
| | lane 5 | | | | | | | NO: 6 |
| D2-6 | FIG. 5b | 0 | 0 | 0 | 1 | 49.5 | 49.5 | Forward |
| | lane 6 | | | | | | | inner |
| | | | | | | | | primer: |
| | | | | | | | | SEQ ID |
| | | | | | | | | NO: 7 |
| | | | | | | | | Backward |
| | | | | | | | | inner |
| | | | | | | | | primer: |
| | | | | | | | | SEQ ID |
| | | | | | | | | NO: 8 |
| D3-1 | FIG. 5c | 0 | 0 | 0 | 0 | 0 | 0 | Forward |
| | lane 1 | | | | | | | outer |
| D3-2 | FIG. 5c | 0 | 0 | 100 | 0 | 0 | 0 | primer: |
| | lane 2 | | | | | | | SEQ ID |
| D3-3 | FIG. 5c | 25 | 25 | 50 | 0 | 0 | 0 | NO: 9 |
| | lane 3 | | | | | | | Backward |
| D3-4 | FIG. 5c | 45 | 45 | 10 | 0 | 0 | 0 | outer |
| | lane 4 | | | | | | | primer: |
| D3-5 | FIG. 5c | 47.5 | 47.5 | 5 | 0 | 0 | 0 | SEQ ID |
| | lane 5 | | | | | | | NO: 10 |
| D3-6 | FIG. 5c | 49.5 | 49.5 | 1 | 0 | 0 | 0 | Forward |
| | lane 6 | | | | | | | inner |
| | | | | | | | | primer: |
| | | | | | | | | SEQ ID |
| | | | | | | | | NO: 11 |
| | | | | | | | | Backward |
| | | | | | | | | inner |
| | | | | | | | | primer: |
| | | | | | | | | SEQ ID |
| | | | | | | | | NO: 12 |

As shown in FIG. 5a, although the DNA templates are extracted from beef mixing with pork and mutton in different ratio, the primer set according to the first embodiment of the invention is still capable of amplifying the target fragment. Moreover, the primer set according to the second embodiment of the invention also can amplify the target fragment in the presence of duck and goose, as shown in FIG. 5b. FIG. 5c shows results of the primer set according to the third embodiment of the invention. The same as FIGS. 5a and 5b, in the presence of beef and mutton, the primer set according to the third embodiment of the invention still can amplify the target fragment even the mixture comprises only 1 wt % of pork (group D3-6.) As a result, the method for identification of meat species according to the embodiments of the invention posses high specificity and is suitable for identification the DNA templates extracted from mixtures.

Trial (E). Identification Test of the Boiled Sample

The DNA templates and the primer sets used in the trail (E) are shown in Table 6. The DNA templates are obtained as followed: 2 grams of raw meat shown in Table 6 are packed in aluminum foils, boiled at 100° C. with a water bath for different time shown in Table 6, and followed by extracting the template DNAs as mentioned above. The method for identification of meat species is the same as above.

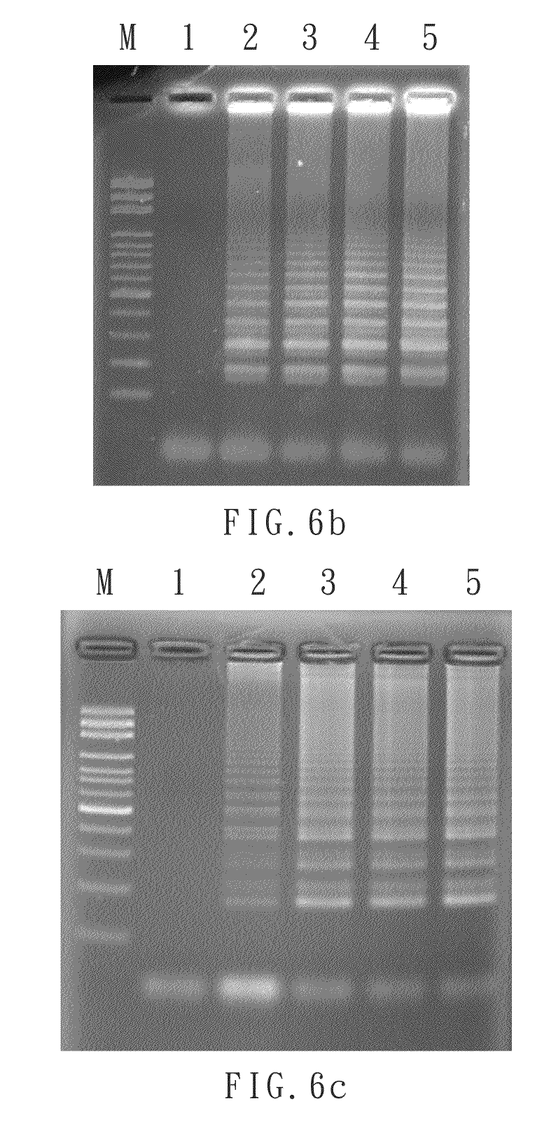

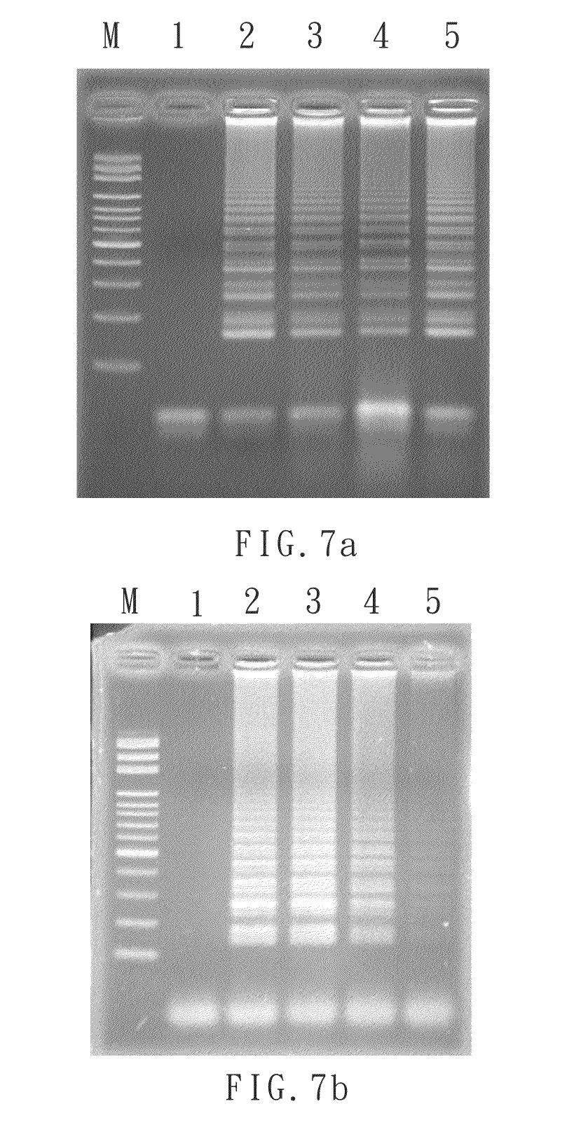

TABLE 6
The DNA templates with different boiled time and the primer
pair used in the trial (E)

| | | DNA templates | | |
|---|---|---|---|---|
| | | | Boiled time | |
| Groups | FIGS. | Sources | (min) | Primer sets |
| E1-1 | FIG. 6a lane 1 | — | 0 | Forward outer |
| E1-2 | FIG. 6a lane 2 | Raw beef | 0 | primer: SEQ ID |
| E1-3 | FIG. 6a lane 3 | Raw beef | 20 | NO: 1 |
| E1-4 | FIG. 6a lane 4 | Raw beef | 40 | Backward outer |
| E1-5 | FIG. 6a lane 5 | Raw beef | 60 | primer: SEQ ID |
| | | | | NO: 2 |
| | | | | Forward inner |
| | | | | primer: SEQ ID |
| | | | | NO: 3 |
| | | | | Backward inner |
| | | | | primer: SEQ ID |
| | | | | NO: 4 |
| E2-1 | FIG. 6b lane 1 | — | 0 | Forward outer |
| E2-2 | FIG. 6b lane 2 | Raw chicken | 0 | primer: SEQ ID |
| E2-3 | FIG. 6b lane 3 | Raw chicken | 20 | NO: 5 |
| E2-4 | FIG. 6b lane 4 | Raw chicken | 40 | Backward outer |
| E2-5 | FIG. 6b lane 5 | Raw chicken | 60 | primer: SEQ ID |
| | | | | NO: 6 |
| | | | | Forward inner |
| | | | | primer: SEQ ID |
| | | | | NO: 7 |
| | | | | Backward inner |
| | | | | primer: SEQ ID |
| | | | | NO: 8 |
| E3-1 | FIG. 6c lane 1 | — | 0 | Forward outer |
| E3-2 | FIG. 6c lane 2 | Raw pork | 0 | primer: SEQ ID |
| E3-3 | FIG. 6c lane 3 | Raw pork | 20 | NO: 9 |
| E3-4 | FIG. 6c lane 4 | Raw pork | 40 | Backward outer |
| E3-5 | FIG. 6c lane 5 | Raw pork | 60 | primer: SEQ ID |
| | | | | NO: 10 |
| | | | | Forward inner |
| | | | | primer: SEQ ID |
| | | | | NO: 11 |
| | | | | Backward inner |
| | | | | primer: SEQ ID |
| | | | | NO: 12 |

Referring to FIG. 6a, lanes 3 to 5, although raw beef is boiled for 20, 40 or 60 minutes, results analyzed by the primer set according to the first embodiment of the invention still show the characteristic ladder pattern. Moreover, the primer set according to the second embodiment of the invention can amplify chicken boiled for 60 minutes, as shown in FIG. 6b. A similar result is observed in FIG. 6c, wherein the primer set according to the third embodiment of the invention can amplify pork boiled for 60 minutes. As a result, by amplifying the target fragments merely being 200 base pairs, the method for identification meat species according to the embodiment of the invention can amplify fragmented DNA templates due to boiling, thereby being suitable for identification of processed food being boiled.

Trial (F). Identification Test of the Pressure-Cooked Sample

The DNA templates and the primer sets used in the trail (F) are shown in Table 7. The DNA templates are obtained as followed: 2 grams of raw meat shown in Table 7 are packed in aluminum foils, pressure-cooked at 121° C., 1.5 atmosphere for different time shown in Table 7, and followed by extracting the template DNAs mentioned above. The method for identification of meat species is the same as above.

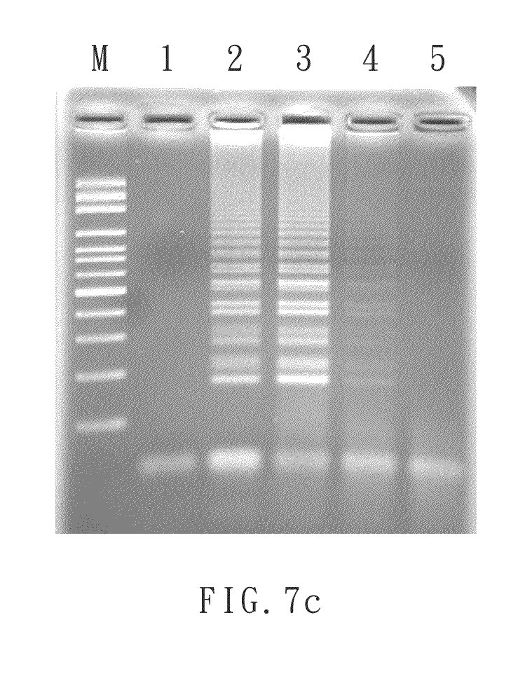

TABLE 7
The DNA templates with different pressure-cooked time and
the primer pair used in the trial (F)

| | | DNA templates | | |
|---|---|---|---|---|
| | | | Pressure- | |
| | | | cooked | |
| Groups | FIGS. | Sources | time (min) | Primer sets |
| F1-1 | FIG. 7a lane 1 | — | 0 | Forward outer |
| F1-2 | FIG. 7a lane 2 | Raw beef | 0 | primer: SEQ ID |
| F1-3 | FIG. 7a lane 3 | Raw beef | 20 | NO: 1 |
| F1-4 | FIG. 7a lane 4 | Raw beef | 40 | Backward outer |
| F1-5 | FIG. 7a lane 5 | Raw beef | 60 | primer: SEQ ID |
| | | | | NO: 2 |
| | | | | Forward inner |
| | | | | primer: SEQ ID |
| | | | | NO: 3 |
| | | | | Backward inner |
| | | | | primer: SEQ ID |
| | | | | NO: 4 |
| F2-1 | FIG. 7b lane 1 | — | 0 | Forward outer |
| F2-2 | FIG. 7b lane 2 | Raw chicken | 0 | primer: SEQ ID |
| F2-3 | FIG. 7b lane 3 | Raw chicken | 20 | NO: 5 |
| F2-4 | FIG. 7b lane 4 | Raw chicken | 40 | Backward outer |
| F2-5 | FIG. 7b lane 5 | Raw chicken | 60 | primer: SEQ ID |
| | | | | NO: 6 |
| | | | | Forward inner |
| | | | | primer: SEQ ID |
| | | | | NO: 7 |
| | | | | Backward inner |
| | | | | primer: SEQ ID |
| | | | | NO: 8 |
| F3-1 | FIG. 7c lane 1 | — | 0 | Forward outer |
| F3-2 | FIG. 7c lane 2 | Raw pork | 0 | primer: SEQ ID |
| F3-3 | FIG. 7c lane 3 | Raw pork | 20 | NO: 9 |
| F3-4 | FIG. 7c lane 4 | Raw pork | 40 | Backward outer |
| F3-5 | FIG. 7c lane 5 | Raw pork | 60 | primer: SEQ ID |
| | | | | NO: 10 |
| | | | | Forward inner |
| | | | | primer: SEQ ID |
| | | | | NO: 11 |
| | | | | Backward inner |
| | | | | primer: SEQ ID |
| | | | | NO: 12 |

Referring to FIG. 7a, the characteristic ladder pattern appears in lane 3, 4 or 5 with the DNA template being extracted from raw beef pressure-cooked for 20, 40 or 60 minutes. Referring to FIG. 7b, the characteristic ladder pattern appears in lane 3 or 4 with the DNA template being extracted from raw chicken pressure-cooked for 20 or 40 minutes.

Further, only a weak, characteristic ladder pattern appears in lane 5 with the DNA template being extracted from raw chicken pressure-cooked for 60 minutes. That is, raw chicken has a limitation of pressure-cooked being 40 minutes. Referring to FIG. 7c, the characteristic ladder pattern appears in lane 3 with the DNA template being extracted from raw pork pressure-cooked for 20 minutes. Only a weak, characteristic ladder pattern appears in lane 4 with the DNA template being extracted from raw pork pressure-cooked for 40 minutes. Moreover, no characteristic ladder pattern appears in lane 5 with the DNA template being extracted from raw pork pressure-cooked for 60 minutes. Therefore, raw pork also has a limitation of pressure-cooked being 40 minutes. As a result, by amplifying the target fragments merely being 200 base pairs, the method for identification meat species according to the embodiment of the invention can amplify the fragmentated DNA templates due to pressure-cooking, thereby being suitable for identification of processed food being pressure-cooked.

Trial (G). Identification Test of the Sample being Commercially Available Processed Food

Commercially available processed food labeled as beef, chicken or pork used in the trail (G) is shown in Table 8. The DNA template is extracted and the method for identification of meat species is the same as above.

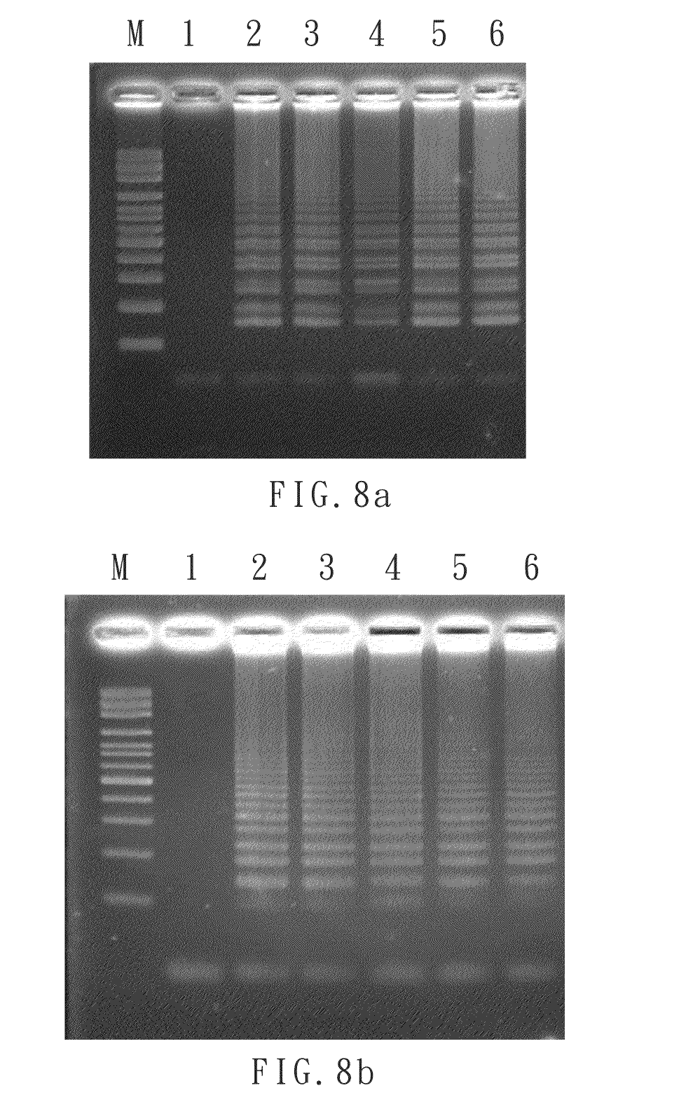

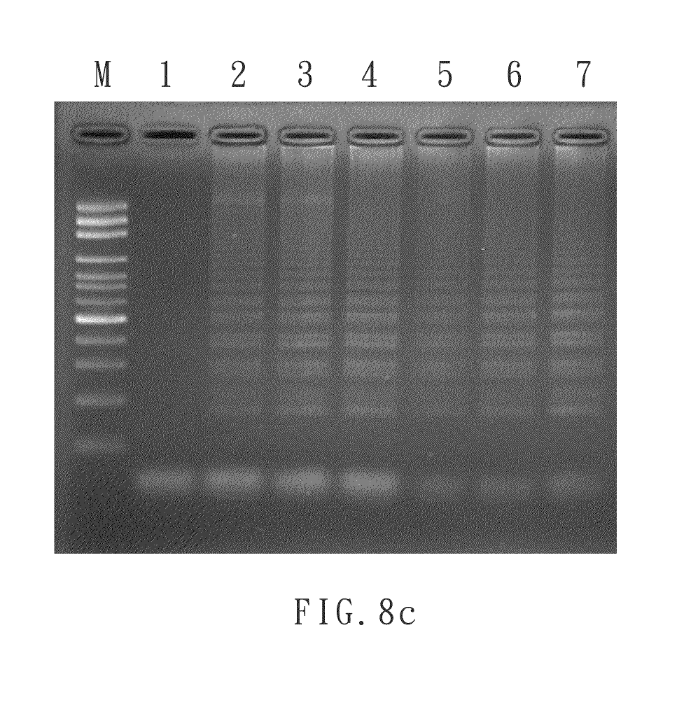

TABLE 8
Sources of the DNA templates and the primer pair used in the
trial (G)

| Groups | FIGS. | Sources | Primer sets |
|---|---|---|---|
| G1-1 | FIG. 8a lane 1 | — | Forward outer |
| G1-2 | FIG. 8a lane 2 | Raw beef | primer: SEQ ID |
| G1-3 | FIG. 8a lane 3 | Beef jerky | NO: 1 |
| G1-4 | FIG. 8a lane 4 | Instant noodles | Backward outer |
| | | (Beef flavor) | primer: SEQ ID |
| G1-5 | FIG. 8a lane 5 | Beef punch | NO: 2 |
| G1-6 | FIG. 8a lane 6 | Cookies | Forward inner |
| | | (Beef flavor) | primer: SEQ ID |
| | | | NO: 3 |
| | | | Backward inner |
| | | | primer: SEQ ID |
| | | | NO: 4 |
| G2-1 | FIG. 8b lane 1 | — | Forward outer |
| G2-2 | FIG. 8b lane 2 | Raw chicken | primer: SEQ ID |
| G2-3 | FIG. 8b lane 3 | Egg pudding | NO: 5 |
| G2-4 | FIG. 8b lane 4 | Chicken floss | Backward outer |
| G2-5 | FIG. 8b lane 5 | Instant noodles | primer: SEQ ID |
| | | (Chicken flavor) | NO: 6 |
| G2-6 | FIG. 8b lane 6 | Chicken sausage | Forward inner |
| | | | primer: SEQ ID |
| | | | NO: 7 |
| | | | Backward inner |
| | | | primer: SEQ ID |
| | | | NO: 8 |
| G3-1 | FIG. 8c lane 1 | — | Forward outer |
| G3-2 | FIG. 8c lane 2 | Raw pork | primer: SEQ ID |
| G3-3 | FIG. 8c lane 3 | Cured meat | NO: 9 |
| G3-4 | FIG. 8c lane 4 | Pork jerky | Backward outer |
| G3-5 | FIG. 8c lane 5 | Pork sausage | primer: SEQ ID |
| G3-6 | FIG. 8c lane 6 | Pork floss | NO: 10 |
| G3-7 | FIG. 8c lane 7 | Instant noodles | Forward inner |
| | | (Pork flavor) | primer: SEQ ID |
| | | | NO: 11 |
| | | | Backward inner |
| | | | primer: SEQ ID |
| | | | NO: 12 |

Referring to FIG. 8a, the characteristic ladder pattern appears in lane 3, 4, 5 or 6 with the DNA template being extracted from commercially available processed food comprising beef Referring to FIG. 8b, the characteristic ladder pattern appears in lane 3, 4, 5 or 6 with the DNA template being extracted from commercially available processed food comprising chicken. Referring to FIG. 8c, the characteristic ladder pattern appears in lane 3, 4, 5, 6 or 7 with the DNA template being extracted from commercially available processed food comprising pork. As a result, the method for identification meat species according to the embodiment of the invention can amplify the DNA template extracted from commercially available processed food.

Accordingly, with the primer set highly specific to the mitochondrial cytochrome b, the method for identification meat species according to the embodiment of the invention is capable of specifically amplifying the target fragments of mitochondrial cytochrome b of beef (Bos indicus), chicken (Gallus gallus) or pork (Sus scrofa domestica). Moreover, by amplifying the target fragments merely being 200 base pairs, the method for identification meat species according to the embodiment of the invention can overcome the problem of unable to specifically amplify the target fragments of the fragmented DNA templates due to processing. Therefore, the method for identification meat species according to the embodiment of the invention has a broader application than the conventional methods.

Moreover, the primer set according to the invention is able to specifically amplify the target fragments of mitochondrial cytochrome b merely being 200 base pairs, thereby overcoming the problem of unable to identify the meat species due to the fragmented DNA template.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. A primer set for identification of meat species comprises:
a pair of outer primers used to amplify a sense strand of target fragment of mitochondrial cytochrome b between positions 51 and 507, comprising a forward outer primer and a backward outer primer, wherein the forward outer primer corresponds to a sense strand of a DNA fragment of mitochondrial cytochrome b between positions 51 and 325, wherein the backward outer primer corresponds to an antisense strand of the DNA fragment of mitochondrial cytochrome b between positions 235 and 507; and
a pair of inner primers comprising a forward inner primer and a backward inner primer, wherein the forward inner primer comprises a first annealing portion and a first warped portion, wherein the first annealing portion corresponds to a sense strand of the DNA fragment of mitochondrial cytochrome b between positions 74 and 360, wherein the first warped portion corresponds to an antisense strand of the DNA fragment of mitochondrial cytochrome b between positions 116 and 401, the backward inner primer comprises a second annealing portion and a second warped portion, wherein the second annealing portion corresponds to a sense strand of the DNA fragment of mitochondrial cytochrome b between positions 153 and 442, wherein the second warped portion corresponds to an antisense strand of the DNA fragment of mitochondrial cytochrome b between positions 116 and 401.

2. The primer set for identification of meat species as claimed in claim 1, with lengths of the forward outer primer and the backward outer primer being 18 to 23 bases.

3. The primer set for identification of meat species as claimed in claim 1, with lengths of the first annealing portion, the first warped portion, the second annealing portion and the second warped portion being 20 to 25 bases.

4. The primer set for identification of meat species as claimed in claim 1, wherein the forward outer primer corresponds to a sense strand of the DNA fragment of mitochondrial cytochrome b of Bos indicus between positions 303 and 325, wherein the backward outer primer corresponds to an antisense strand of the DNA fragment of mitochondrial cytochrome b of Bos indicus between positions 490 and 507, wherein the first annealing portion corresponds to a sense strand of the DNA fragment of mitochondrial cytochrome b of Bos indicus between positions 336 and 360, wherein the first warped portion corresponds to an antisense strand of the DNA fragment of mitochondrial cytochrome b of Bos indicus between positions 377 and 401, wherein the second annealing portion corresponds to a sense strand of the DNA fragment of mitochondrial cytochrome b of Bos indicus between positions 423 and 442, wherein the second warped portion corresponds to an antisense strand of the DNA fragment of mitochondrial cytochrome b of Bos indicus between positions 464 and 483.

5. The primer set for identification of meat species as claimed in claim 4, with the forward outer primer having a sequence set forth in SEQ ID NO: 1, with the backward outer primer having a sequence set forth in SEQ ID NO: 2, with the forward inner primer having a sequence set forth in SEQ ID NO: 3, with the backward inner primer having a sequence set forth in SEQ ID NO: 4.

6. The primer set for identification of meat species as claimed in claim 1, wherein the forward outer primer corresponds to a sense strand of the DNA fragment of mitochondrial cytochrome b of Gallus gallus between positions 70 and 88, wherein the backward outer primer corresponds to an antisense strand of the DNA fragment of mitochondrial cytochrome b of Gallus gallus between positions 241 and 259, wherein the first annealing portion corresponds to a sense strand of the DNA fragment of mitochondrial cytochrome b of Gallus gallus between positions 100 and 119, wherein the first warped portion corresponds to an antisense strand of the DNA fragment of mitochondrial cytochrome b of Gallus gallus between positions 140 and 161, wherein the second annealing portion corresponds to a sense strand of the DNA fragment of mitochondrial cytochrome b of Gallus gallus between positions 168 and 189, wherein the second warped portion corresponds to an antisense strand of the DNA fragment of mitochondrial cytochrome b of Gallus gallus between positions 215 and 234.

7. The primer set for identification of meat species as claimed in claim 6, with the forward outer primer having a sequence set forth in SEQ ID NO: 5, with the backward outer primer having a sequence set forth in SEQ ID NO: 6, with the forward inner primer having a sequence set forth in SEQ ID NO: 7, with the backward inner primer having a sequence set forth in SEQ ID NO: 8.

8. The primer set for identification of meat species as claimed in claim 1, wherein the forward outer primer corresponds to a sense strand of the DNA fragment of mitochondrial cytochrome b of Sus scrofa domestica between positions 51 and 68, wherein the backward outer primer corresponds to an antisense strand of the DNA fragment of mitochondrial cytochrome b of Sus scrofa domestica between positions 235 and 255, wherein the first annealing portion corresponds to a sense strand of the DNA fragment of mitochondrial cytochrome b of Sus scrofa domestica between positions 74 and 97, wherein the first warped portion corresponds to an antisense strand of the DNA fragment of mitochondrial cytochrome b of Sus scrofa domestica between positions 116 and 140, wherein the second annealing portion corresponds to a sense strand of the DNA fragment of mitochondrial cytochrome b of Sus scrofa domestica between positions 153 and 177, wherein the second warped portion corresponds to an antisense strand of the DNA fragment of mitochondrial cytochrome b of Sus scrofa domestica between positions 210 and 229.

9. The primer set for identification of meat species as claimed in claim 8, with the forward outer primer having a sequence set forth in SEQ ID NO: 9, with the backward outer primer having a sequence set forth in SEQ ID NO: 10, with the forward inner primer having a sequence set forth in SEQ ID NO: 11, with the backward inner primer having a sequence set forth in SEQ ID NO: 12.

10. A method for identification of meat species comprising:
providing a primer set as claimed in claim 1;
mixing DNA template, a reaction buffer, dNTPs, the primer set and a Bst DNA polymerase to obtain a reaction mixture;
amplifying the target fragment of mitochondrial cytochrome b of the DNA template of the reaction mixture at 65° C. to obtain an amplified target fragment; and
analyzing the amplified target fragment by agarose electrophoresis.

11. The method for identification of meat species as claimed in claim 10, with the forward outer primer having a sequence set forth in SEQ ID NO: 1, with the backward outer primer having a sequence set forth in SEQ ID NO: 2, with the forward inner primer having a sequence set forth in SEQ ID NO: 3, with the backward inner primer having a sequence set forth in SEQ ID NO: 4.

12. The method for identification of meat species as claimed in claim 10, with the forward outer primer having a sequence set forth in SEQ ID NO: 5, with the backward outer primer having a sequence set forth in SEQ ID NO: 6, with the forward inner primer having a sequence set forth in SEQ ID NO: 7, with the backward inner primer having a sequence set forth in SEQ ID NO: 8.

13. The method for identification of meat species as claimed in claim 10, with the forward outer primer having a sequence set forth in SEQ ID NO: 9, with the backward outer primer having a sequence set forth in SEQ ID NO: 10, with the forward inner primer having a sequence set forth in SEQ ID NO: 11, with the backward inner primer having a sequence set forth in SEQ ID NO: 12.

14. The method for identification of meat species as claimed in claim 10, wherein the target fragment is amplified at 65° C. for 1 hour.

15. The method for identification of meat species as claimed in claim 10, with the DNA template having amounts set forth in 1 to 100 ng.

16. The method for identification of meat species as claimed in claim 10, wherein the primer set comprises the pair of outer primers being 0.5 μM and the pair of inner primers being 4 μM.
